(19) United States
(12) Patent Application Publication
(10) Pub. No.: US 2007/0288051 A1
(43) Pub. Date: Dec. 13, 2007

(54) Fluid-filled cervical dilator

(76) Inventors: Bruce Beyer, Nashville, TN (US); George Bikakis, Potomac, MD (US); Drew Lansdown, Chicago, IL (US); John Moustoukas, Metairie, LA (US); Michael Nichols, Nashville, TN (US)

Correspondence Address: MILES & STOCKBRIDGE PC, 1751 PINNACLE DRIVE, SUITE 500, MCLEAN, VA, 22102-3833, US

(21) Appl. No.: 11/785,425

(22) Filed: Apr. 17, 2007

Related U.S. Application Data
(60) Provisional application No. 60/792,351, filed on Apr. 17, 2006.

(51) Int. Cl. A61M 29/02

(52) U.S. Cl. 606/193

(57) ABSTRACT

A cervical canal dilator comprising an elongate tubular or cylindrical shaft having a distal end and a proximal end; the interior of the shaft being provided with internal cavities that communicate with anchor and dilation balloons in such a manner as to permit the separate inflation thereof; the anchor balloon being positioned on the distal end of the shaft and being capable of anchoring the dilator against the bottom of the cervix when inflated after the dilator is inserted in a cervix and the remaining dilation balloons being positioned between the distal and proximal ends so as to effect optimum dilation of the cervical canal when inflated after the device is inserted and anchored in place by inflation of the anchor balloon.

### BACKGROUND OF INVENTION

In obstetric procedures, a pregnant patient may present with the complication of a cervix that cannot dilate on its own or, for example, in the case of overdue delivery owing to an insufficient opening of the cervix which may be caused by insufficient rotation of the fetus's head or by abnormal delivery because of the fetus's size, steps must be taken to effect a delivery which will not injure the mother's body or the fetus by dilating the cervix artificially. Current methods to remedy this problem include the insertion of metal rods (see, e.g., U.S. Pat. No. 5,207,702) of increasing diameter over time, the use of an absorbing material that expands in the presence of fluid (see, e.g., U.S. Pat. No. 4,624,258), or the use of prostaglandins (see, e.g., U.S. Pat. No. 4,237,888). These current methods all have undesirable consequences. For the metal rods, the procedure requires constant attention from a physician, and there is also a risk of damaging the cervix. The absorbing material, often Japanese seaweed, Laminariais not readily controllable and there is a risk of fragmentation. As with any chemical process, the prostaglandins present the potential for unpredictable and harmful side effects from the drugs.

Specific systems currently in use for dilating the cervix are generally as follows: Hegar's dilator, Bossi's dilator colpeurynter, half colpeurynter, folding type of colpeurynter, metreurynter, dilator of metal spring type, Bougie, and the method of inserting gauze into the cervix.

These methods, however, are not overly effective nor efficient in dilating the cervix and are difficult to use. Accordingly, injury to the mother's body and to the fetus often occur. For example, Bossi's dilator, and the dilator of the metal spring type are apt to injure the cervix of the mother's body. The structure of the colpeurynter and metreurynter types cause difficulty in inserting them into the uterus. In the use of these instruments, the position of the fetus is apt to be changed suddenly from the occipital position to the sacral position, thereby pressing against the fetus' head. Further, in the use of the colpeurynter, it is usual to expand the device by pouring water into it after it is inserted into the uterus and then to pull it by weight at the end of the tube. In this procedure, however, often the device slips off outside of the uterus and dilation of the cervix cannot then be obtained.

The use of elastic balloons, made of materials such as latex rubber or silicone, is well established in medicine, typically for applications where low pressures are needed for fixation and occlusion.

Elastic balloons are inflated by volume and can typically stretch 100-600%. They do not retain well-defined shapes, though, and cannot be used to exert high pressure in medical applications. When the pressure is released, elastic balloons recover close to their original size and shape. An example of an elastic balloon is seen with Foley catheters that are passed through the urethra into the bladder; an elastic balloon located near the distal tip is inflated to occlude the entrance to the bladder and the catheter lumen can evacuate urine from the bladder.

Cervical canal dilators having tubular shafts with inflatable expanding members, such as balloons, are well known and have functions ranging from incontinence catheters to assisting in childbirth. One or more balloons are inflated after positioning the dilator through the cervical canal. The expanded balloons secure the tubular shaft in position and, in combination with the function of dilation, frequently provide the critical passageway for fluid passage, diagnostic devices, or treatment instruments.

Devices and processes for dilation prior to medical procedures are described in U.S. Pat. Nos. 3,848,602; 4,664,114; 5,104,377; 5,947,991. Despite historical uses of inelastic balloons in medicine, there have been limitations in the use of these balloons for dilation of the cervical canal. This challenge arises due to the lack of visualization of the cervix to assist the user in placing the catheter, whereas in vascular applications such as angioplasty the catheter placement can be visualized using fluoroscopy. For this reason, a single balloon for dilation, such as those used in angioplasty, are ineffective, resulting in a potential to either insert the catheter too far, causing damage to the uterus, or fail to dilate the full length of the cervix, if the catheter is not placed far enough into the cervix. Prior attempts to overcome this lack of visualization through the use of multiple balloons or unique balloon shapes have been limited by the unique problems with cervical dilation occurring because there is more resistance to dilation of the internal os (portion of the cervical canal adjacent to the uterus) which is furthest from the operator, than the portion of the cervix closer to the vagina. The unequal resistance tends to push a balloon out toward the vagina, so patients may not have their inner os properly dilated. Innovations to past design ideas are required to overcome this issue.

One proposed design utilizes an inelastic balloon that has two sections, the proximal section cylindrical and the distal balloon ellipsoidal in shape. In the single balloon embodiment, the ellipsoidal portion of the balloon is assumed to inflate before the cylindrical section based on the lack of external pressure in the uterus. The feature assumes the catheter is placed correctly and that the uterus is flexible and uniform. The inelastic ellipsoidal balloon is intended to act as an anchor and to dilate the internal os, but use of an ellipsoidal balloon to inflate the internal os may result in an overdilation of the internal os, risking damage to the cervix including an incompetent cervix (a cervix unable to remain closed for a fetus causing miscarriage).

Another proposed design contains separate cylindrical and ellipsoidal balloons, both made of inelastic material. If the ellipsoidal balloon is used as an anchor for positioning, use of this design results in under-inflation of the inner os because inelastic balloons have a taper to allow the folding of the balloon when deflated. The taper of the balloons results in a set gap between the two balloons at the point of the inner os, resulting in an under-inflated portion of the cervix. If the catheter is alternately pulled so the ellipsoidal balloon is located at the point of the inner os, the inner os may alternately be overinflated, as with the single balloon design. Another problem encountered with the use of inelastic balloons is that the balloons tend to be fragile. This does not pose a problem in vascular applications, since they are shipped with protective sheathing and passed through plastic catheter introducers without the use of metal instrumentation. In gynecological uses, it may be necessary to use several traditional metal instruments, including tenaculums.

### SUMMARY OF THE INVENTION

The device of the invention comprises plural, preferably, three elastic balloons affixed to a shaft of any desired rigidity or flexibility for insertion through the vaginal canal into the cervical canal as shown in the drawings. The cervical dilator of the present invention solves the problems associated with the prior all cervical dilators by allowing dilation of the cervix over longer periods of time at slow and controlled rates, as determined by an external pressure source (not shown and does not comprise part of the invention). The use of fluid, e.g., saline renders the device safe to use in the body, since the introduction of saline during a malfunction will not damage the body.

### DETAILED DESCRIPTION OF THE INVENTION

The device comprises plural, preferably, three elastic balloons as shown in the drawings. The balloons may be constructed of any suitable biocompatible elastic material such as polyurethane, for example. The device is inserted so that the top-most, anchor balloon is above the cervix, and the top balloon is filled with saline from a pump, for example, to inflate and expand the top-most balloon beyond the effective diameter of the upper cervix to anchor the device in the cervical canal. Then, over a period of several hours, the other two balloons, or dilation balloons, are filled with saline from the pump. Two separate catheters or chambers are provided in the interior of the shaft of the device that allow for separate inputs of fluid into the top and remaining balloons, respectively. It will be appreciated by those skilled in the all that any suitable, biocompatible fluid (gas or liquid) may be employed to inflate the balloons.

The device of the invention is preferably connected to pump(s) which are operated by a controlled (computerized, for example) system that automatically monitors expansion of the balloons to dilate the cervical canal according to a predetermined schedule thereby eliminating any requirement for supervision by a physician beyond the initial insertion of the device into the cervical canal and once the dilation process has commenced. The device and method of the invention hold great potential in the marketplace, where 15% of all labors are induced and at least half of these require one of the existing cervical dilation methods.

The device of the invention provides alternative systems that are safer, more effective and efficient than conventional cervical dilation systems and methods. They allow more precise control of the rate of dilation, as well as requiring less direct supervision by medical staff. The device presents no adverse effects to the patient and also helps make the dilation procedure more accommodating for the patient.

The device may be constructed according to the dimensions set forth in the drawings.

The cervical canal dilator of the present invention comprises an elongate tubular or cylindrical shaft having a distal end and a proximal end. The interior of the shaft is provided with internal cavities that communicate with the anchor and dilation balloons in such a manner as to permit the separate inflation thereof. The anchor balloon is positioned on the distal end of the shaft whereas the remaining dilation balloons are positioned between the distal and proximal ends so as to effect optimum dilation of the cervical canal when inflated after the device is anchored in place by inflation of the anchor balloon.

### BRIEF DESCRIPTION OF THE DRAWINGS

FIG. 1 is an elevational view of the cervical dilator of the invention

FIGS. 2 and 3 are cross-sectional views of the cervical dilator of the invention in place uninflated and inflated in the cervix of a patient, respectively

FIG. 4 is a cross-sectional view of the cervical dilator of the invention

FIGS. 5-7 are graphical depictions of the cervical dilator of the invention showing relevant dimensions thereof.

### DETAILED DESCRIPTION

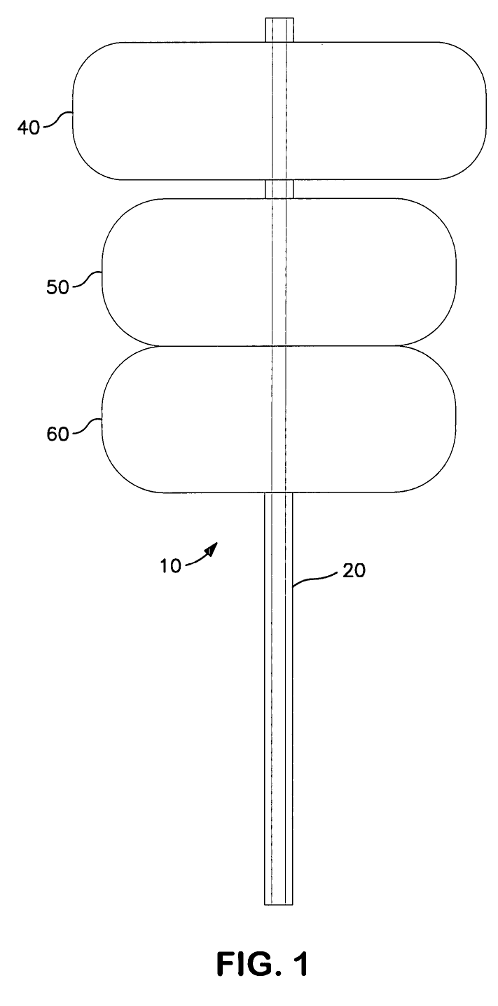

Referring now in specific detail to the drawings in which like referenced numerals identify similar or identical elements throughout the several views, and initially to FIG. 1, a novel cervical canal dilator assembly 10 is shown having a shaft 20, an inflatable anchor balloon 40 and inflatable cervical canal dilator balloons 50 and 60. Cervical canal dilator 10, has a distal end 12 and a proximal end 14.

Shaft 20 is a solid shaft 20 defining inner walls for a first inner cavity 25, a second inner cavity 27, and a third inner cavity 29. Inner cavity 25 is provided with a port 26 for fluid communication with anchor balloon 40. Similarly, inner cavities 27 and 29 are provided with ports 28 and 30 for communication with inflatable dilator balloons 50 and 60. It will be understood by those skilled in the art that dilator balloons 50 and 60 may be inflated simultaneously by injecting therein fluid through ports 28 and 30 via the same pump system (not shown) or separately inflated at different times utilizing separate pump or similar systems (not shown).

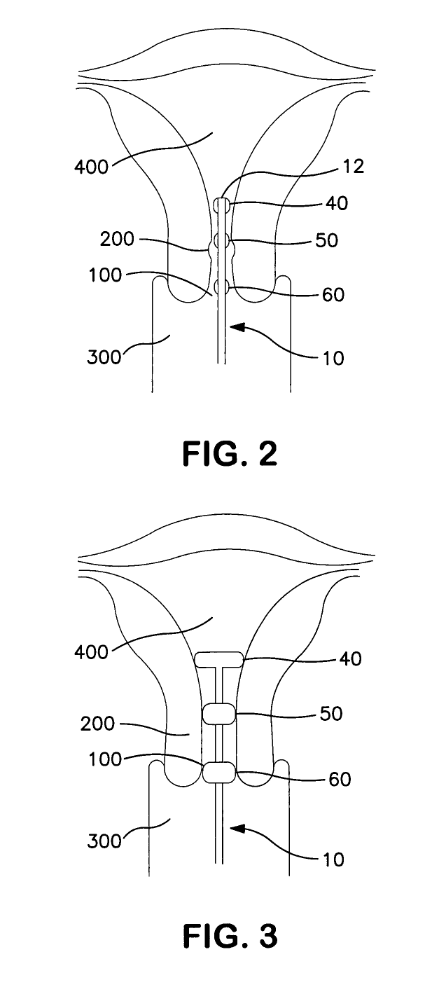

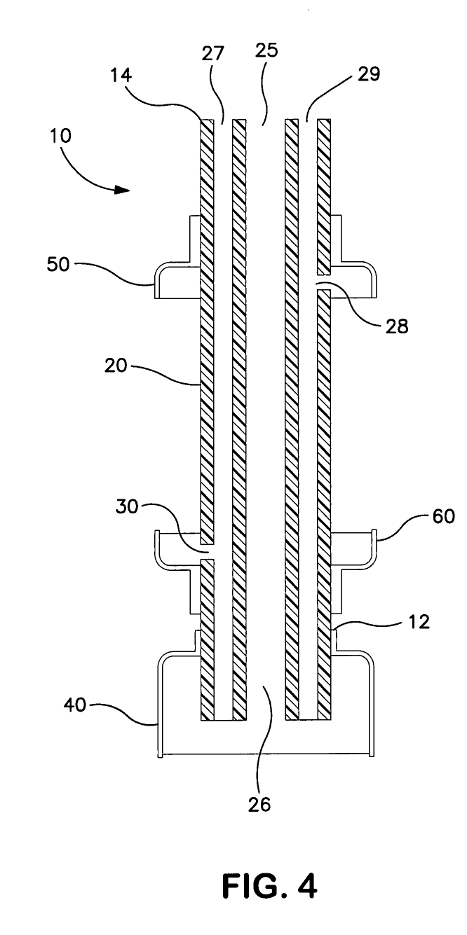

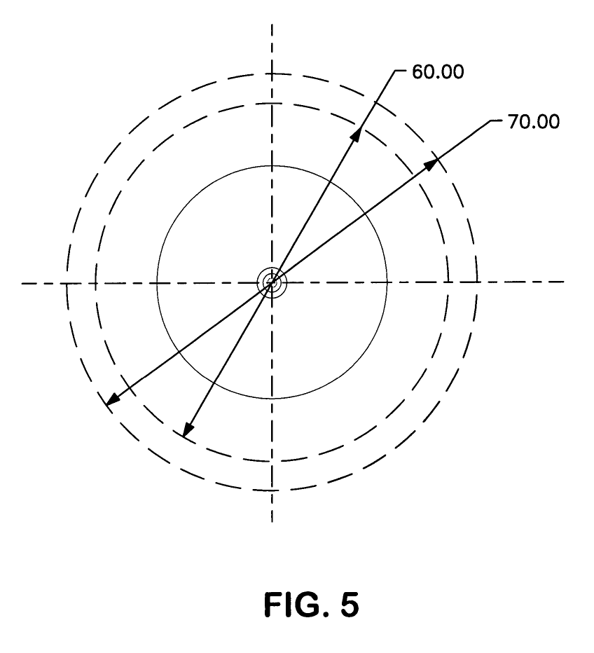

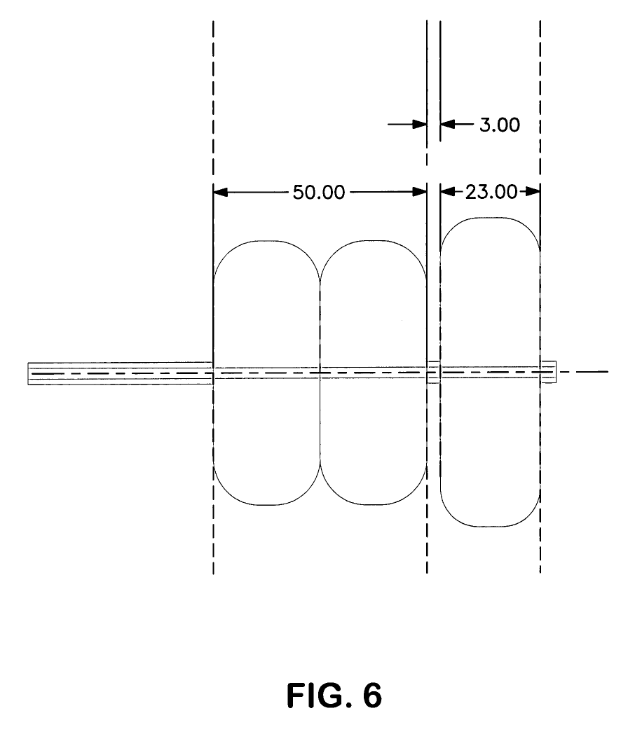

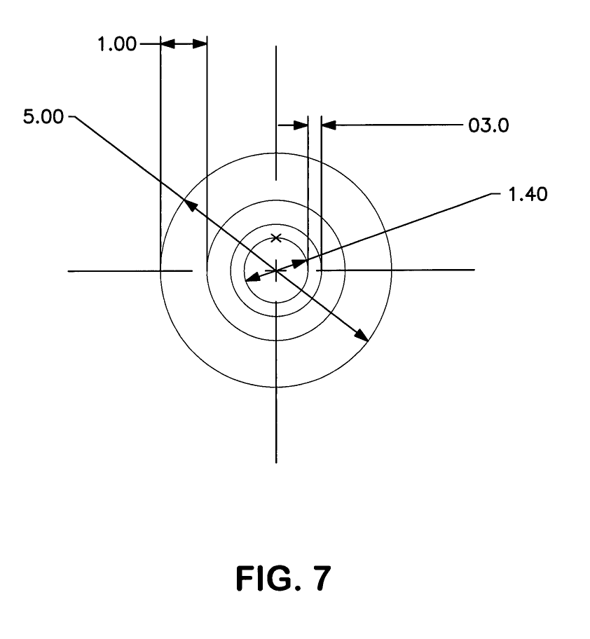

In operation, the physician selects a desired configuration of cervical canal dilator 10 for application with the patient for the dilation of the patient's cervical canal to the predetermined maximum diameter. This process includes evaluating the patient internal geometries. Referring to FIGS. 2 and 3 dilator 10 is positioned at least partially into the cervical opening 100 of cervix 200 via vaginal canal 300. Once distal end 12 has been inserted a predetermined distance to position anchor balloon 40 within the Uterus 400, a predetermined volume of fluid, such as, but not limited to a saline solution, is injected to inflate member 40 and engage the internal edge of cervix. Dilator balloons 50 and 60 are then inflated separately or simultaneously to the desired predetermined diameters to dilate the cervical canal 200. Inflation of members 50 and 60 is typically done in a series of graduated steps.

Although the illustrative embodiments of the present disclosure have been described herein with reference to the accompanying drawings, it is to be understood that the disclosure is not limited to those precise embodiments, and that various other changes and modifications may be affected therein by one skilled in the art without departing from the scope or spirit of the disclosure. All such changes and modifications are intended to be included within the scope of the disclosure.

What is claimed is:

1. A cervical canal dilator comprising an elongate tubular or cylindrical shaft having a distal end and a proximal end; the interior of the shaft being provided with internal cavities that communicate with anchor and dilation balloons in such a manner as to permit the separate inflation thereof; the anchor balloon being positioned on the distal end of the shaft and being capable of anchoring the dilator against the bottom of the cervix when inflated after the dilator is inserted in a cervix and the remaining dilation balloons being positioned between the distal and proximal ends so as to effect optimum dilation of the cervical canal when inflated after the device is inserted and anchored in place by inflation of the anchor balloon.

2. The cervical canal dilator of claim 1, wherein said internal cavities are positioned so as to be capable of inflating said dilator balloons simultaneously.

3. The cervical canal dilator of claim 1, wherein said internal cavities are positioned so as to be capable of inflating said dilator balloons separately.

4. A method of dilating a cervical canal of a patient, comprising the steps of inserting the dilator of claim 1 into the cervix of said patient through the vaginal canal, inflating the anchor balloon to position the inflated anchor balloon against the inner side of the cervical canal, and inflating said dilator balloons to dilate the cervical canal of the patient.
